(19) United States
(12) Patent Application Publication
(10) Pub. No.: US 2013/0108676 A1
(43) Pub. Date: May 2, 2013

(54) USE OF A CELLULOSE FIBER

(71) Applicants: Sigrid Redlinger, Lenzing (AT); Gert Kroner, Lenzing (AT)

(72) Inventors: Sigrid Redlinger, Lenzing (AT); Gert Kroner, Lenzing (AT)

(73) Assignee: LENZING AKTIENGESELLSCHAFT, Lenzing (AT)

(21) Appl. No.: 13/636,180

(22) PCT Filed: Mar. 15, 2011
(86) PCT No.: PCT/EP2011/053875

§ 371 Date: Jan. 18, 2013

(30) Foreign Application Priority Data
Mar. 25, 2010 (EP) 10157765.8

(51) Int. Cl. A61K 8/02; A61Q 19/00

(52) U.S. Cl. CPC A61K 8/0216; A61Q 19/00
USPC 424/401; 424/443

(57) ABSTRACT

The present invention relates to the use of a cellulose fibre containing incorporated a chitosan and/or chitosan salt and/or having a chitosan and/or chitosan salt on its surface, for the preparation of an article that, when being used, is in contact with the skin of a living mammal with a mean pressure p of 0.001 N/cm²≦p<0.24 N/cm², wherein the cellulose fibre is provided at least in a part of those areas of the article that, when being used, are in contact with the skin.

### BACKGROUND OF THE INVENTION

### Field of the Invention

The invention relates to the use of cellulose fibres modified with chitosan.

“Cellulose fibres modified with chitosan” are to be understood for the purpose of the present invention as cellulose fibres containing incorporated a chitosan and/or chitosan salt and/or having a chitosan and/or chitosan salt on their surface. Fibres featuring a chitosan (or a chitosan salt) on their surface are in the following designates as fibres “impregnated with chitosan”.

Chitosan consists of poly-(1,4)-2-amino-2-desoxy-beta-D-glucose and is prepared by means of deacetylation of chitin (poly-(1,4)-2-acetamide-2-desoxy-beta-D-glucose). Due to reasons of solubility—chitin is not soluble in water, organic solvents, diluted acids and bases—chitosan, which is soluble in diluted acids, aqueous methanol and glycerine, is of significantly greater importance.

Cellulose fibres modified with chitosan are generally known:

Viscose fibres with incorporated chitin/chitosan are e.g. available under the trade name Crabyon (company Omikenshi Co) and Chitopoly (company Fuji Spinning Co). These fibres are, for instance, prepared by dispersing chitosan or acetylated chitosan in powder form with a corn size of less than 10 μm in an amount of 0.5 to 2 w % in water and adding it to the viscose spinning solution (U.S. Pat. No. 5,320,903). Consequently, the fibres are fabricated according to the common viscose or polynosic method. Socks for people suffering from diabetes, which contain fibres with the trade name “Crabyon”, are commercially available.

Further methods of preparation for chitosan-incorporated viscose fibres are described in the publication U.S. Pat. No. 5,756,111 (complex pre- and post dissolution processes at cryogenic temperature in order to obtain alkaline chitin-chitosan solutions for addition to the viscose solution), U.S. Pat. No. 5,622,666 (addition of micro-crystalline chitosan and a natural polymer soluble in water and/or alkali, e.g. sodium alginate, which may form ionic bounds to the chitosan, as a dispersion to the viscose spinning solution), and PCT/FI90/00292 and FI 78127 (addition of micro-crystalline chitosan to the spinning mass), respectively.

In DE 195 44 097 there is described a method for the preparation of moulded bodies from polysaccharide mixtures by dissolution of cellulose and another second polysaccharide in an organic polysaccharide solvent miscible with water (preferably NMMO), which may also contain a second solvent.

Furthermore, there is described in KR-A 9614022 the preparation of chitin-cellulose fibres, also designated as “chitulose”, by dissolving chitin and cellulose in a solvent pertaining to the group of dimethyl imidazoline/LiCl, dichloro acetate/chlorinated hydrocarbon, dimethyl acetamide/LiCl, N-methyl pyrrolidone/LiCl and consequently preparing yarns in the wet spinning method.

In EP-A 0 883 645 there is claimed, among others, the addition of chitosan to the solution as a modified compound in order to increase the smoothness of casings for foodstuff. The modified compounds have to be miscible with the solution of cellulose/NMMO/water.

KR-A-2002036398 describes the incorporation of chitosan derivatives with quaternary ammonium groups, which are rather difficult to produce, in fibres.

DE-A 100 07 794 describes the preparation of polymer compositions, comprising a biodegradable polymer and a material from sea plants and/or the shell of sea animals as well as the preparation of moulded bodies thereof. There is also claimed the addition of material from sea plants, sea animals in powder form, powder suspension or liquid form to the cellulose solution prepared according to the Lyocell method.

Furthermore, there is described in the final report “Erzeugnisse aus Polysaccharidverbunden” (Taeger, E.; Kramer, H.; Meister, F.; Vorwerg, W.; Radosta, S; TITK—Thüringisches Institut für Textil- und Kunststoff-Forschung, 1997, p. 1-47, Report-Nr. FKZ 95/NR 036 F) that chitosan is dissolved in diluted organic and inorganic acids and then precipitated in an aqueous NMMO solution. In this way, there is obtained a suspension of fine chitosan crystals in the cellulose solution, which is then subjected to a spinning process.

Further methods for the preparation of cellulose fibres modified with chitosan are described in WO 2004/007818, WO 2009/092121 as well as PCT/AT2009/000334 (not pre-published).

EP 1 243 688 describes the use of chitosan-nanoparticles for the preparation of fibres, yarns, knitted fabrics or textile fabrics. Nano-chitosans are approximately spherical solid bodies with a mean diameter in the range from 10 to 300 nm, which may be embedded between the fibrilles due to their small particle diameter. The preparation of nano-chitosanes is carried out by means of spray drying, evaporation technique or the expansion of supercritical solutions.

In WO 01/32751 there is described a method for the preparation of nano-particulate chitosan for cosmetic and pharmaceutical preparations with a size diameter of 10 to 1000 nm, wherein the pH of an aqueous acid chitosan solution is increased in the presence of a surfactant to a level so that the chitosan will precipitate. Furthermore, there is described in WO 91/00298 the preparation of micro-crystalline chitosan dispersions and powders with a particle diameter of 0.1 to 50 μm, wherein the pH of an aqueous acid chitosan solution is increased to a level so that the chitosan will precipitate.

WO 97/07266 describes the treatment of a Lyocell fibre with a 0.5% acetic chitosan solution.

AT 8388 U describes the use of a cellulose fibre, which incorporates a chitosan or a chitosan salt and/or has a chitosan or a chitosan salt on its surface, in a non-woven textile and/or an absorbent hygiene product.

It is generally known to provide textile articles with functional substances, e.g. cosmetic agents.

The measures proposed in this regard include the introduction of micro-capsules containing a substance into the textile article.

WO 09/124686 A describes the modification of a textile article with so-called “micro-spheres”, which are, in contrast to micro-capsules, not destroyed when being used and which are described to be “re-chargeable” with the substance.

DE 199 40 239 A describes a bandage for caring or curing uses, at the internal side of which there may be exchangeably fixed a therapeutically effective layer.

Further textile articles treated with substances or cosmetic agents are, for example, described in US 2004/082239A, WO 06/106546 A, and WO 06/068418 A.

With the so far proposed textiles thus modified, there has not been demonstrated so far an actually positive effect, and the textiles have the disadvantage that the efficacy, this is the functionality of the introduced substance has to be reconstituted (“re-charged”) on a regular basis.

In this connection, it is state of the art in the fields of medicine and cosmetics to characterize the condition of the skin by means of the following parameters: skin moisture, transepidermal water loss, skin elasticity, skin surface profile, skin roughness, depth of wrinkles, microscopic assessment, pH of the skin, skin flora swabs. These parameters are measured, e.g. in order to provide for the evaluation of the effect of a cosmetic treatment.

For the parameters skin moisture and transepidermal water loss, there are in particular to be determined areas evaluating the skin conditions as follows:

| Skin moisture in % | Skin condition |
|---|---|
| <30% | Very dry |
| 30-40% | dry |
| >40% | Normal |
| >60% | Damaged by occlusion, |
| | swelling of the horny layer |

| Transepidermal water loss g/m²/h | |
|---|---|
| 3-9 | Normal skin with normally functioning |
| | barrier function |
| 710-18 | Slightly damaged skin |
| >25 | Damaged skin with clearly damaged |
| | barrier function |

A particular need is therefore manifested for textiles, which are worn close to the body, in particular skin tight and with a particular pressure onto the skin, this is elastic and optionally also body shaping textiles. It has been known that textiles that are worn rather close to the body do exert influence on the main parameters. By covering the skin with textiles under normal conditions of wear, skin moisture tends to decrease, and the transepidermal water loss increases. It is generally known that textiles, prepared from fibres with rather low water absorption capacity/water steam permeability, such as polyester, cause a so-called occlusion, this is blocking the discharge of water and heat via the skin and as a consequence swelling of the horny layer, which damages the skin barrier function and increases the transepidermal water loss of the skin, although it simultaneously increases the moisture level of the skin. This effect will result, even with initially healthy skin, in damage (irritation dermatosis), and in the case of already damaged or dry skin, this effect is even more significant. In an ideal way, a textile that is worn close to the body shall not have any negative influence on the above mentioned skin parameters.

The present invention aims at providing an article, which, when being used, is in contact with the skin of a living mammal with a pressure p of 0.001 N/cm²≦p<0.24 N/cm², and which has a positive influence on the skin, in particular on parameters like, e.g. skin moisture and transepidermal water loss.

This aim is solved by the use of a cellulose fibre, which contains incorporated a chitosan and/or chitosan salt and/or has a chitosan and/or a chitosan salt on its surface, for the preparation of such an article, wherein the cellulose fibre is provided in at least a part of those areas of the article that, when being used, are in contact with the skin.

It has surprisingly been found that textiles made from cellulose fibres modified with chitosan do not exert any negative influence on the above mentioned main parameters but that these parameters are rather even positively influenced thereby. It has been found that, depending on the initial state of the healthy skin, the transepidermal water loss will remain constant or will be decreased if a textile having cellulose fibres modified with chitosan is worn close to the body; at the same time, however, skin moisture will be maintained, and skin roughness will be reduced.

Maintaining the natural micro moisture film on the skin due to the presence of cellulose fibres modified with chitosan will also have the effect that the elasticity of the skin will be maintained, and the skin condition on a microscopic level will be improved. Furthermore, also the flora condition of the skin will not be affected.

In the case of damaged skin, additionally there will be promoted the generation of new skin cells.

Preferably, the article in which the cellulose fibre modified with chitosan is used, is a textile article.

The textile article is especially preferably selected from the group consisting of corsetry, bed linen, sheets, nightwear, e.g. pyjamas or nightdresses, underwear, socks, pillows, leggings, jeans, t-shirts, sleeping masks, body shaping garments, shoe insert, bandages, mull, elastic bandages, support bandages, and jersey tube bandages, wherein, when being used, there is applied onto the skin a mean pressure p of 0.001 N/cm²≦p<0.24 N/cm².

The pressure p, on the one hand, may be applied by the article itself, namely if this is provided with an elastic construction. The extent of the pressure applied will depend on the textile construction and the type of the elastic material used, respectively.

This pressure may be measured similarly to the methods generally known for medicinal compression stockings, e.g. Deutsches Institut für Gütesicherung und Kennzeichnung, Medical Compression Hosiery Quality Assurance RAL-GZ 387/1, examination by the HOSY measurement device (Institut Hohenstein). Alternatively, the pressure may also be measured directly at the individual by means of commercially available pressure sensors, e.g. Kikuhime pressure pad sensor by the company TT MediTrade ApS or piezo-resististive pressure probes of the type MCDM, producer MIPM Mammendorf.

In the doctoral dissertation “Produktentwicklung für körpernahe Bekleidung unter Berücksichtigung der textilen Materialeigenschaften” by Dipl.- Ing. Tünde Kirstein, Faculty for Mechanical Engineering at the Technical University Dresden, there are described further features of garments worn close to the body as well as methods for measurement.

On the other side, pressure p, in the range as defined according to the invention, may also be applied, if, for example, during recumbency, there is exerted pressure onto the sleeping layer (sheet) or onto a garment like a pyjama. Also in these uses and applications, the use of a cellulose fibre modified with chitosan will prove favourable. A preferred area of application are anti-decubitus articles, e.g. anti-decubitus mattresses, cushions, map layers, sheets, etc.

The contact pressure exerted by recumbent persons may, for example, be measured by means of pressure sensors, e.g. piezo-resistive pressure recorders.

In order to take into account local pressure peaks (e.g. with recumbent persons in the area of the hips), there is measured, in the majority of the mentioned test methods, the pressure at different sites, on the basis of which the mean pressure will be determined. The pressure range defined according to the invention relates to this mean pressure, as far as areas with different pressures in the respective articles may be present.

For the mean pressure p of the article onto the skin, the following applies: 0.01 N/cm²≦p, preferably 0.05 N/cm²≦p, particularly preferably 0.1 N/cm²≦p.

According to the invention, the cellulose fibres modified with chitosan may also be used in articles, which are commonly not considered “textile”, which come, however, into contact with the skin under a particular pressure, e.g. the internal side of plaster bandages.

The cellulose fibre used according to the invention is preferably a man-made cellulose fibre. In particular preferable in this connection is the use of cellulose fibres, selected from the group consisting of viscose fibres, polynosic fibres, Modal fibres and Lyocell fibres as well as mixtures thereof.

Viscose fibres are fibres, which are obtained from an alkaline solution of the cellulose xanthogenate (viscose) by means of precipitation and regeneration of the cellulose.

Modal fibres are cellulose fibres, which are characterized, according to the definition issued by the BISFA, by a high level of wet strength and a high wet module (the force necessary in order to expand a fibre in its wet condition by 5%).

The generic name “Lyocell” was issued by the BISFA (The International Bureau for the Standardisation of Man Made Fibres) and stands for cellulose fibres, which are prepared from cellulose solutions in an organic solvent. Solvents preferably used are tertiary amine oxides, in particular N-methyl-morpholine-N-oxide (NMMO). A method for preparing Lyocell fibres is e.g. described in U.S. Pat. No. 4,246,221.

An especially preferred cellulose fibre is a Lyocell fibre with a content of chitosan of 0.1 w % and more, preferably 0.2 w % to 1 w %, in particular preferable 0.4 w % to 0.6 w %. There has been shown that a Lyocell fibre with a chitosan content of as low as 0.1 to 1% already has a cosmetic effect, and in addition, as has been described in the PCT/AT2009/000334 (not pre-published), also has a wound healing promoting effect. The positive influence on the skin cells, however, will be decreased in the case of a higher content of chitosan (in particular with contents of significantly more than 1 w %). Surprisingly, there is present in a Lyocell fibre modified with chitosan also with a rather low content of 0.1 w % to 1 w %, a synergistic effect in regard to both the cosmetic effect, i.e. for the cosmetic treatment of the skin, as well as the positive influence on the skin cells, e.g. for the improvement of wound healing and reduction of the scarring phenomenon.

The cellulose fibre modified with chitosan may be preferably prepared according to a method described in WO 2004/007818, WO 2009/092121 as well as PCT/AT2009/000334 (not pre-published).

PCT/AT2009/000334 describes a method for treating a cellulosic moulded body, wherein the moulded body is contacted with an acid solution of a chitosan, characterized in that the chitosan has a deacetylation degree of at least 80%, a nitrogen content of at least 7 w %, preferably at least 7.5 w %, %, a weight average molecular weight M_{w} (D) of 10 kDa to 1000 kDa, preferably 10 kDa to 160 kDa and a viscosity of 1 w % solution in 1 w % acetic acid at 25° C. of 1000 mPas or less, preferably 400 mPas or less, particularly preferably 200 mPas or less. The moulded bodies are provided in particular in the form of fibres and for treatment, preferably in never-dried form.

The cellulose fibre modified with chitosan may be provided in mixture with other fibres, e.g. cotton, polyester, elastane and polyamide. In particular for elastic articles, it is necessary to add an elastic (fibre) material.

The preparation of elastic articles, in particular in the textile field, is well-known for those skilled in the art.

The article may have a structure consisting of several layers, wherein the cellulose fibre modified with chitosan is to be provided on the side of the article intended for being in contact with the skin.

The present invention also relates to an article for contacting the skin of a living mammal with a mean pressure p of 0.001 N/cm²≦p<0.24 N/cm², containing at least in a part of those areas of the article, which, when being used, are in contact with the skin, a cellulose fibre containing incorporated a chitosan and/or chitosan salt and/or having a chitosan and/or chitosan salt on its surface.

The above given explanations and descriptions in regard to the details of the article and the cellulose fibre used therefore are also effective for the article according to the invention.

The present invention further relates to the use of the article according to the invention for the cosmetic treatment of the skin as well as wound healing and reduction of the scarring phenomenon.

### EXAMPLES

Four test persons were wearing for one week, for at least 10 hours over night tailor-made and close-fitting arm warmers, which cover the arm from the wrist up beyond the elbow and apply a mean pressure of 0.02 N/cm². On the last day, these arm warmers were not taken off, but only for measurement. The arm warmers were not washed while being used. The test persons were not allowed to put cream on their arms, and they were only allowed to wash their arms with water. There was carried out a comparison between left and right arm. The region of measurement was the inside of the forearm.

The materials used were Single Jersey made from polyester (PES) and Lyocell fibre (trademark “Tencel”), respectively, and Lyocell fibre “Tencel C” (=Lyocell fibre with 0.4% chitosan coating), respectively.

| | Left side | Right side |
|---|---|---|
| Test person 1 | Tencel | Tencel C |
| Test person 2 | Tencel | Tencel C |
| Test person 3 | PES | Tencel |

Before the wear test, the moisture of the skin and the transepidermal water loss=TEWL were measured. The measurement was performed after 30 minutes of air-conditioning at 24° C./50% humidity. Furthermore, there was carried out a skin flora swab at the inside of the forearm.

After the wear test, the sleeve was taken off in the controlled climate chamber, and after 30 minutes of air-conditioning, the moisture of the skin and the TEWL were measured, and a skin flora swab was performed on the inside of the forearm.

### Results:

| Test person 1 | Tencel | % change | Tencel C | % change |
|---|---|---|---|---|
| TEWL initial value | 11.95 g/m2/h | | 16.03 g/m2/h | |
| TEWL after 7 days | 14.39 g/m2/h | +20.4% | 14.44 g/m2/h | −20% |
| Skin moisture | 43.6% | | 36.1% | |
| Skin moisture after 7 | 38.5% | −11.7% | 34.3% | −4.9% |
| days | | | | |

| Test person 2 | Tencel | % change | Tencel C | % change |
|---|---|---|---|---|
| TEWL initial value | 10.28 | | 11.38 | |
| TEWL after 7 days | 11.77 | +14.5% | 11.65 | 0% |
| Skin moisture | 32.8 | | 37.6 | |
| Skin moisture after 7 | 24.6 | −25% | 30.5 | −18.8% |
| days | | | | |

| Test person 3 | Polyester | % change | Tencel | % change |
|---|---|---|---|---|
| TEWL initial value | 4.86 | | 5.57 | |
| TEWL after 7 days | 7.3 | +50% | 6.23 | +11.8% |
| Skin moisture | 35 | | 34.4 | |
| Skin moisture after 7 | 37.5 | +7.1% | 32.3 | −6.3% |
| days | | | | |

1. A process for preparing an article comprising providing a cellulose fiber which comprises incorporated chitosan and/or chitosan salt and/or having a chitosan and/or chitosan salt on its surface, wherein said article, when in use, is in contact with the skin of a living mammal with a mean pressure p of 0.001 N/cm²≦p<0.24 N/cm², and wherein the cellulose fiber is provided at least in a part of those areas of the article that, when in use, are in contact with the skin.

2. The process according to claim 1, wherein the article is a textile article selected from the group consisting of corsetry, bed linen, sheets, nightwear, underwear, socks, pillows, leggings, jeans, t-shirts, sleeping masks, body shaping garments, shoe inserts, bandages, mull, elastic bandages, support bandages, and jersey tube bandages.

3. The process according to claim 1, wherein the article is elastic.

4. The process according to claim 1, wherein the cellulose fiber is a man-made fiber selected from the group consisting of viscose fibers, polynosic fibers, Modal fibers and Lyocell fibers and mixtures thereof.

5. The process according to claim 4, wherein the cellulose fiber is a Lyocell fiber with a content of chitosan of at least 0.1 w %.

6. The process according to claim 1, wherein for the mean pressure p of the article applied onto the skin the following applies: 0.01 N/cm²≦p.

7. An article for contacting the skin of a living mammal with a mean pressure p of 0.001 N/cm²≦p<0.24 N/cm², comprising at least in a part of those areas of the article which, when in use, are in contact with the skin, a cellulose fiber which comprises incorporated chitosan and/or chitosan salt and/or has a chitosan and/or chitosan salt on its surface.

8. The article according to claim 7, wherein the article is in the form of a textile article selected from the group consisting of corsetry, bed linen, sheets, nightwear, underwear, socks, pillows, leggings, jeans, t-shirts, sleeping masks, body shaping garments, shoe inserts, bandages, mull, elastic bandages, support bandages, and jersey tube bandages.

9. The article according to claim 8, wherein the article is elastic.

10. The article according to claim 7, wherein the cellulose fiber is a man-made cellulose fiber selected from the group consisting of viscose fibers, polynosic fibers, Modal fibers and Lyocell fibers and mixtures thereof.

11. The article according to claim 10, wherein the cellulose fiber is a Lyocell fiber with a content of chitosan of at least 0.1 w %.

12. The article according to claim 7, wherein for the mean pressure p of the article onto the skin the following applies: 0.01 N/cm²≦p.

13. A cosmetic treatment of the skin comprising the article according to claim 7.

14. A treatment for wound healing and/or reduction of scarring comprising the article according to claim 7.

15. The process according to claim 2, wherein the nightwear is selected from the group consisting of pyjamas and nightdresses.

16. The process according to claim 5, wherein the Lyocell fiber has a content of chitosan of 0.2 w % to 1 w %.

17. The process according to claim 16, wherein the Lyocell fiber has a content of chitosan of 0.4 w % to 0.6 w %.

18. The process according to claim 6, wherein for the mean pressure p of the article applied onto the skin the following applies: 0.05 N/cm²≦p.

19. The process according to claim 18, wherein for the mean pressure p of the article applied onto the skin the following applies: 0.1 N/cm²≦p.

20. The article according to claim 8, wherein the nightwear is selected from the group consisting of pyjamas and nightdresses.

21. The article according to claim 11, wherein the Lyocell fiber has a content of chitosan of 0.2 w % to 1 w %.

22. The article according to claim 21, wherein the Lyocell fiber has a content of chitosan of 0.4 w % to 0.6 w %.

23. The article according to claim 12, wherein for the mean pressure p of the article onto the skin the following applies: 0.05 N/cm²≦p.

24. The article according to claim 23, wherein for the mean pressure p of the article onto the skin the following applies: 0.1 N/cm²≦p.
